(19) United States
(12) Patent Application Publication
(10) Pub. No.: US 2025/0235261 A1
(43) Pub. Date: Jul. 24, 2025

(54) PLASMA CREATION VIA NONAQUEOUS OPTICAL BREAKDOWN OF LASER PULSE ENERGY FOR BREAKUP OF VASCULAR CALCIUM

(71) Applicants: Boston Scientific Scimed, Inc., Maple Grove, MN (US); Bolt Medical, Inc., Carlsbad, CA (US)

(72) Inventors: Daniel Massimini, Brooklyn Park, MN (US); Roger McGowan, Otsego, MN (US); Haiping Shao, Plymouth, MN (US); Christopher A. Cook, Laguna Niguel, CA (US)

(21) Appl. No.: 19/172,037

(22) Filed: Apr. 7, 2025

Related U.S. Application Data
(63) Continuation of application No. 18/346,122, filed on Jun. 30, 2023.
Application No. 18/346,122 is a continuation-in-part of application No. 16/874,065, filed on May 14, 2020, now Pat. No. 11,717,139.
(60) Provisional application No. 62/965,069, filed on Jan. 23, 2020.
(60) Provisional application No. 62/863,506, filed on Jun. 19, 2019.

(51) Int. Cl. A61B 18/24; A61B 18/00

(52) U.S. Cl. CPC A61B 18/245; A61B 2018/00065; A61B 2018/00285; A61B 2018/00404

(57) ABSTRACT

A catheter system (100) for treating a treatment site (106) within or adjacent to a blood vessel (108) includes a power source (124), a light guide (122) and a plasma target (242). In various embodiments, the light guide (122) receives power from the power source (124). The light guide (122) has a distal tip (244), and the light guide (122) emits light energy (243) in a direction away from the distal tip (244). The plasma target (242) is spaced apart from the distal tip (244) of the light guide (122) by a target gap distance (245). The plasma target (242) is configured to receive light energy (243) from the light guide (122) so that a plasma bubble (234) is generated at the plasma target (242). The power source (124) can be a laser and the light guide (122) can be an optical fiber. The catheter system (100) can also an inflatable balloon (104) that encircles the distal tip (244) of the light guide (122). The plasma target (242) can be positioned within the inflatable balloon (104). The plasma target (242) can have a target face (1672) that receives the light energy (243) from the light guide (122). The plasma target (242) can be formed from one or more of tungsten, tantalum, platinum, molybdenum, niobium, iridium, magnesium oxide, beryllium oxide, tungsten carbide, titanium nitride, titanium carbonitride and titanium carbide.

### RELATED APPLICATIONS

The present application is a continuation application of and claims priority on U.S. patent application Ser. No. 18/346,122, filed on Jun. 30, 2023, and entitled “PLASMA CREATION VIA NONAQUEOUS OPTICAL BREAKDOWN OF LASER PULSE ENERGY FOR BREAKUP OF VASCULAR CALCIUM”. Additionally, U.S. patent application Ser. No. 18/346,122 is a continuation-in-part application claiming the benefit under 35 U.S.C. 120 on U.S. patent application Ser. No. 16/874,065 (now U.S. Pat. No. 11,717,139), filed on May 13, 2020. Further, U.S. patent application Ser. No. 16/874,065 claims priority on U.S. Provisional Application Ser. No. 62/863,506, filed on Jun. 19, 2019, and on U.S. Provisional Application Ser. No. 62/965,069, filed on Jan. 23, 2020. To the extent permitted, the contents of U.S. patents application Ser. Nos. 18/346,122 and 16/874,065 (now U.S. Pat. No. 11,717,139), and U.S. Provisional Application Ser. Nos. 62/863,506 and 62/965,069 are incorporated in their entirety herein by reference.

### BACKGROUND

Vascular lesions within and adjacent to vessels in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, and vascular graft bypass, to name a few. Such interventions may not always be ideal or may require subsequent treatment to address the lesion.

Creation of a plasma via optical breakdown of an aqueous solution requires a significant amount of energy in a short amount of time, upon which it is converted into a therapeutic bubble and/or a therapeutic pressure wave. With sufficiently high energy and short pulse durations, there is potential to damage a distal end of a light guide used to deliver light energy to generate the plasma. A means to enhance the conversion efficiency of the light energy to (plasma) pressure wave and bubble growth would reduce the required power handling requirements of the optical delivery system. Therefore, less input energy would be required for an equivalent therapy while minimizing potential damage to the light guide.

Creation of the plasma near the distal end of a small diameter light guide as in the case of aqueous optical breakdown as one method for an intravascular lithotripsy catheter has the potential for self-damage due to its proximity to the plasma creation and/or the pressure wave, high plasma temperatures, and waterjet from collapse of the bubble, as non-exclusive examples.

### SUMMARY

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

The present invention is directed toward a catheter system for treating a treatment site within or adjacent to a blood vessel. In certain embodiments, the catheter system includes a power source, a light guide, and a plasma target. The light guide receives power from the power source. The light guide has a distal tip, and the light guide emits light energy in a direction away from the distal tip. The plasma target is spaced apart from the distal tip of the light guide by a target gap distance. The plasma target is configured to receive light energy from the light guide so that a plasma is generated at the plasma target upon receiving the light energy from the light guide.

In some embodiments, the power source is a laser. In various embodiments, the light guide is an optical fiber.

In certain embodiments, the catheter system can also include an inflatable balloon that encircles the distal tip of the light guide.

In various embodiments, the catheter system can also include an inflatable balloon. In some such embodiments, the plasma target can be positioned within the inflatable balloon.

In some embodiments, the target gap distance is greater than 1 μm, 10 μm, 100 μm, 1 mm, 2 mm, 3 mm, 5 mm, and/or 1 cm.

In various embodiments, the plasma target can have a substantially circular cross-sectional configuration, a substantially square cross-sectional configuration, a substantially rectangular cross-sectional configuration, a substantially oval cross-sectional configuration, a substantially pentagonal cross-sectional configuration, a substantially hexagonal cross-sectional configuration, a substantially octagonal cross-sectional configuration, a polygonal cross-sectional configuration, a parallelogram cross-sectional configuration, a trapezoidal cross-sectional configuration or a substantially diamond-shaped cross-sectional configuration.

In certain embodiments, the catheter system can also include a guidewire lumen. In some such embodiments, the light guide can be coupled to the guidewire lumen.

In some embodiments, the plasma target has a target face that receives the light energy from the light guide. In various embodiments, the target face has an angle that is substantially orthogonal relative to a direction the light energy is emitted to the plasma target. In various embodiments, the target face has an angle that is greater than approximately 45 degrees and less than approximately 135 degrees relative to a direction the light energy is emitted to the plasma target. In certain embodiments, the target face can have an angle that is greater than zero degrees and less than 180 degrees relative to a direction the light energy is emitted to the plasma target.

In various embodiments, the light guide includes a distal region having a longitudinal axis. The direction the light energy is emitted can be substantially along the longitudinal axis of the distal region. Alternatively, the direction the light energy is emitted can be substantially perpendicular to the longitudinal axis of the distal region. Still alternatively, the direction the light energy is emitted can be angled relative to the longitudinal axis of the distal region. For example, in some embodiments, the direction the light energy is emitted has an angle relative to the longitudinal axis that is greater than zero degrees and less than 180 degrees. In various embodiments, the direction the light energy is emitted can have an angle relative to the longitudinal axis that is greater than 45 degrees and less than 135 degrees.

In certain embodiments, the catheter system can include a plurality of plasma targets that are spaced apart from the distal tip of the light guide. In some such embodiments, at least one of the plurality of plasma targets can be configured to receive light energy from the light guide.

In various embodiments, the plasma target can be at least partially formed from one of stainless steel and its variants, tungsten, tantalum, platinum, molybdenum, niobium, and iridium.

In some embodiments, the plasma target can be at least partially formed from one of magnesium oxide, beryllium oxide, tungsten carbide, titanium nitride, titanium carbonitride, and titanium carbide.

In certain embodiments, the plasma target can be at least partially formed from one of diamond CVD and diamond.

In various embodiments, the plasma target can be at least partially formed from a transition metal, a metal alloy, and/or a ceramic material.

In some embodiments, the plasma target can be fixedly coupled to the light guide. Alternatively, the plasma target can be movably coupled to the light guide.

Still alternatively, the plasma target can be uncoupled from the light guide.

In some applications, the catheter system can include a guidewire lumen. In some such embodiments, the plasma target can substantially encircle the guidewire lumen.

In certain embodiments, the target face can include one or more surface features, which can include one or more of an indentation, a projection, and a beveled edge.

In some embodiments, the target face can have a conical configuration, a pyramidal configuration, a dome-shaped configuration, a concave configuration, a convex configuration, a multi-faceted configuration, a coiled configuration, a spring-like configuration, and/or a somewhat spiral configuration.

In various embodiments, the plasma target can be movable relative to the light guide. In some embodiments, the plasma target can be spring-loaded.

In certain embodiments, the catheter system can include a guidewire lumen, and the plasma target can be secured or otherwise coupled to the guidewire lumen.

In some embodiments, the catheter system can include a second light guide that receives power from the power source. The second light guide can have a second distal tip. The second light guide can emit light energy in a direction away from the second distal tip toward the plasma target. The plasma target can be spaced apart from the second distal tip of the second light guide. The plasma target can be configured to receive light energy from the second light guide so that a second plasma is generated at the plasma target upon receiving the light energy from the second light guide.

In certain embodiments, the catheter system can include a second light guide and a second plasma target. The second light guide can receive power from the power source. The second light guide can have a second distal tip. The second light guide can emit light energy in a direction away from the second distal tip toward the second plasma target. The second plasma target can be spaced apart from the plasma target and the second distal tip of the second light guide. The second plasma target can be configured to receive light energy from the second light guide so that a second plasma is generated at the second plasma target upon receiving the light energy from the second light guide.

In various embodiments, the present invention can also be directed toward a method for creating plasma to optically break up vascular calcium in a blood vessel using laser pulse energy. In certain embodiments, the method includes the step of providing any one of the catheter systems shown and/or described herein.

In some embodiments, the plasma target can be formed at least partially from a polymer, a polymeric material, and/or a plastic such as polyimide and nylon.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view of a catheter system having features of the present invention in accordance with various embodiments herein;

FIG. 2 is a simplified schematic side view of one embodiment of a portion of the catheter system, including one embodiment of a portion of a catheter;

FIG. 3A is a simplified schematic side view of one embodiment of a portion of the catheter system, including another embodiment of a portion of the catheter, shown in an inflated state;

FIG. 3B is a simplified schematic side view of the portion of the catheter illustrated in FIG. 3A, shown in a deflated state;

FIG. 4 is a simplified schematic side view of one embodiment of a portion of the catheter system, including another embodiment of a portion of the catheter;

FIG. 5 is a simplified schematic side view of one embodiment of a portion of the catheter system, including another embodiment of a portion of the catheter;

FIG. 6 is a simplified schematic side view of one embodiment of a portion of the catheter system, including another embodiment of a portion of the catheter;

FIG. 7A is a simplified schematic side view of one embodiment of a portion of the catheter system, including another embodiment of a portion of the catheter;

FIG. 7B is a simplified schematic side view of one embodiment of a portion of the catheter system, including another embodiment of a portion of the catheter;

FIG. 8 is a schematic cross-sectional view of the catheter system taken on line 8-8 in FIG. 1;

FIG. 9 is a schematic cross-sectional view of another embodiment of the catheter system;

FIG. 10 is a schematic cross-sectional view of yet another embodiment of the catheter system;

FIG. 11 is a schematic cross-sectional view of still another embodiment of the catheter system;

FIG. 12 is a schematic cross-sectional view of a portion of the catheter system including one embodiment of a distal portion of a light guide;

FIG. 13 is a schematic cross-sectional view of a portion of the catheter system including an embodiment of the distal portion of the light guide;

FIG. 14 is a schematic cross-sectional view of a portion of the catheter system including another embodiment of the distal portion of the light guide;

FIG. 15 is a schematic cross-sectional view of a portion of the catheter system including yet another embodiment of the distal portion of the light guide;

FIG. 16 is a simplified schematic side view of a portion of one embodiment of the catheter, including an embodiment of a portion of a plasma target;

FIGS. 16A-16J are cross-sectional views of various embodiments of the plasma target taken on line 16-16 in FIG. 16;

FIGS. 17A-17H are perspective views of various embodiments of a portion of the plasma target having a target face;

FIG. 18 is a cross-sectional view of a portion of the catheter system including one embodiment of a portion of the catheter;

FIG. 19 is a cross-sectional view of a portion of the catheter system including another embodiment of a portion of the catheter;

FIG. 20 is a cross-sectional view of a portion of the catheter system including another embodiment of a portion of the catheter; and

FIG. 21 is a cross-sectional view of a portion of the catheter system including another embodiment of a portion of the catheter.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DESCRIPTION

Treatment of vascular lesions can reduce major adverse events or death in affected subjects. A major adverse event is one that can occur anywhere within the body due to the presence of a vascular lesion. Major adverse events can include, but are not limited to major adverse cardiac events, major adverse events in the peripheral or central vasculature, major adverse events in the brain, major adverse events in the musculature, or major adverse events in any of the internal organs.

In various embodiments, the systems and methods disclosed herein describe the use of a catheter systems including any number of light guides for generating pressure waves within an inflatable balloon (sometimes referred to herein simply as “balloon”) for disrupting intervascular lesions. The catheter systems herein can utilize light energy to generate a plasma near the light guide disposed in the inflatable balloon located at or near a treatment site. As used herein, the treatment site can include a vascular lesion such as a calcified vascular lesion or a fibrous vascular lesion (hereinafter sometimes referred to simply as a “lesion”), typically found in a blood vessel and/or a heart valve. The plasma formation can initiate a pressure wave and can initiate the rapid formation of one or more bubbles that can rapidly expand to a maximum size and then dissipate through a cavitation event that can also launch a pressure wave upon collapse. The rapid expansion of the plasma-induced bubbles can generate one or more pressure waves within a balloon fluid and thereby impart pressure waves upon the treatment site. The pressure waves can transfer mechanical energy through an incompressible balloon fluid to a treatment site to impart a fracture force on the lesion. Without wishing to be bound by any particular theory, it is believed that the rapid change in balloon fluid momentum upon a balloon wall of the inflatable balloon that is in contact with or positioned near the lesion is transferred to the lesion to induce fractures in the lesion.

The catheter systems can include a catheter configured to advance to the lesion located within or adjacent to the blood vessel, where the catheters include a catheter shaft. The catheters also include one or more light guides disposed along the catheter shaft and within a balloon. Each light guide can be configured to be in optical communication with a light and/or power source.

Those of ordinary skill in the art will realize that the following detailed description of the present invention is illustrative only and is not intended to be in any way limiting. Other embodiments of the present invention will readily suggest themselves to such skilled persons having the benefit of this disclosure. Additionally, other methods of delivering energy to the lesion can be utilized, including, but not limited to, electric current-induced plasma generation. Reference will now be made in detail to implementations of the present invention, as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

As an overview, in certain embodiments, the light guides can be configured to include one or more diverting features configured to direct light to exit from the light guide toward a side surface of the light guide and toward the balloon wall. The diverting features can direct light to exit in a direction away from the axis of the light guide, or in an off-axis direction. Additionally, or in the alternative, the light guides can each include one or more light windows disposed along the longitudinal or axial surfaces of each light guide and in optical communication with a diverting feature. The light windows can include a portion of the light guide that allows light to exit the light guide from within the light guide, such as a portion of the light guide lacking a cladding material on or about the light guide. The inflatable balloons described herein can be coupled to the catheter shaft and/or other structures, and can be inflated with a balloon fluid.

The inflatable balloon can include a balloon wall and can be configured to expand from a deflated state suitable for advancing the catheter through a patient's vasculature to an inflated state suitable for anchoring the catheter in position relative to a treatment site. The power source can be configured to provide sub-millisecond pulses of a light from the power source to initiate plasma formation in a balloon fluid within the balloon to cause rapid bubble formation and to impart pressure waves upon the treatment site.

Various embodiments of this invention shine laser light energy onto a plasma target to cause plasma generation via interaction with plasma target material rather than optical breakdown of the balloon fluid thereby moving the plasma creation away from a distal end of the optical fiber (light guide). This can be accomplished by positioning the plasma target away from the distal end of the optical fiber to absorb the light energy and convert it into a plasma at some distance away from the distal end of the light guide.

As used herein, the terms “intravascular lesion” and “vascular lesion” are used interchangeably unless otherwise noted.

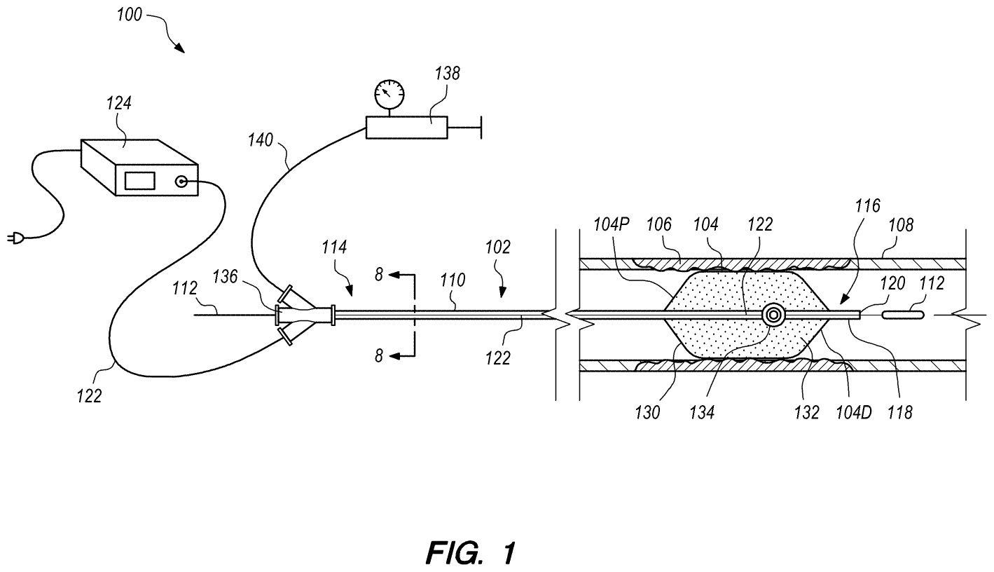

It is appreciated that the catheter systems herein can include many different forms. Referring now to FIG. 1, a schematic cross-sectional view is shown of a catheter system in accordance with various embodiments herein. A catheter system 100 is suitable for imparting pressure to induce fractures in a vascular lesion within or adjacent a vessel wall of a blood vessel. In the embodiment illustrated in FIG. 1, the catheter system 100 can include one or more of a catheter 102, one or more light guides 122, a power source 124, a manifold 136, and a fluid pump 138.

The catheter 102 includes an inflatable balloon 104 (sometimes referred to herein as “balloon”). The catheter 102 is configured to move to a treatment site 106 within or adjacent to a blood vessel 108. The treatment site 106 can include a vascular lesion such as a calcified vascular lesion, for example. Additionally, or in the alternative, the treatment site 106 can include a vascular lesion such as a fibrous vascular lesion.

The catheter 102 can include the balloon 104, a catheter shaft 110, and a guidewire 112. The balloon can be coupled to the catheter shaft 110. The balloon can include a balloon proximal end 104P and a balloon distal end 104D. The catheter shaft 110 can extend between a shaft proximal end 114 and a shaft distal end 116. The catheter shaft 110 can include a guidewire lumen 118 which is configured to move over the guidewire 112. The catheter shaft 110 can also include an inflation lumen (not shown). In some embodiments, the catheter 102 can have a distal end opening 120 and can accommodate and be moved over and/or along the guidewire 112 so that the balloon 104 is positioned at or near the treatment site 106.

The catheter shaft 110 of the catheter 102 can encircle one or more light guides 122 (only one light guide 122 is illustrated in FIG. 1 for clarity) in optical communication with a power source 124. The light guide 122 can be at least partially disposed along and/or within the catheter shaft 110 and at least partially within the balloon 104. In various embodiments, the light guide 122 can be an optical fiber, and the power source 124 can be a laser. The power source 124 can be in optical communication with the light guide 122. In some embodiments, the catheter shaft 110 can encircle multiple light guides, such as a second light guide, a third light guide, etc.

The balloon 104 can include a balloon wall 130. The balloon 104 can expand from a collapsed configuration suitable for advancing at least a portion of the catheter shaft 102 through a patient's vasculature to an expanded configuration suitable for anchoring the catheter 102 into position relative to the treatment site 106. The power source 124 of the catheter system 100 can be configured to provide sub-millisecond pulses of light from the power source 124, along the light guide 112, to a location within the balloon 104. The pulses of light, resulting in light energy, thereby induce plasma formation in a balloon fluid 132 within the balloon 104. The plasma formation causes rapid bubble formation, and imparts pressure waves upon the treatment site 106. Exemplary plasma-induced bubbles are shown as bubbles 134 in FIG. 1. The balloon fluid 132 can be a liquid or a gas. As provided in greater detail herein, the plasma-induced bubbles 134 are intentionally formed at some distance away from the light guide 122 so that the likelihood of damage to the light guide is decreased.

In various embodiments, the sub-millisecond pulses of light can be delivered to near the treatment site 106 at a frequency of from at least approximately 1 hertz (Hz) up to approximately 5000 Hz. In some embodiments, the sub-millisecond pulses of light can be delivered to near the treatment site 106 at a frequency from at least 30 Hz to 1000 Hz. In other embodiments, the sub-millisecond pulses of light can be delivered to near the treatment site 106 at a frequency from at least 10 Hz to 100 Hz. In yet other embodiments, the sub-millisecond pulses of light can be delivered to near the treatment site 106 at a frequency from at least 1 Hz to 30 Hz. In some embodiments, the sub-millisecond pulses of light can be delivered to near the treatment site 106 at a frequency that can be greater than or equal to 1 Hz, 2 Hz, 3 Hz, 4 Hz, 5 Hz, 6 Hz, 7 Hz, 8 Hz, or 9 Hz, 10 Hz, 20 Hz, 30 Hz, 40 Hz, 50 Hz, 60 Hz, 70 Hz, 80 Hz, 90 Hz, 100 Hz, 200 Hz, 300 Hz, 400 Hz, 500 Hz, 600 Hz, 700 Hz, 800 Hz, 900 Hz, 1000 Hz, 1250 Hz, 1500 Hz, 1750 Hz, 2000 Hz, 2250 Hz, 2500 Hz, 2750 Hz, 3000 Hz, 3250 Hz, 3500 Hz, 3750 Hz, 4000 Hz, 4250 Hz, 4500 Hz, 4750 Hz, or 5000 Hz or can be an amount falling within a range between any of the foregoing. Alternatively, the sub-millisecond pulses of light can be delivered to near the treatment site 106 at a frequency that can be greater than 5000 Hz.

It is appreciated that the catheter system 100 herein can include any number of light guides 122 in optical communication with the power source 124 at the proximal portion 114, and with the balloon fluid 132 within the balloon 104 at the distal portion 116. For example, in some embodiments, the catheter system 100 herein can include from one light guide 122 to five light guides 122. In other embodiments, the catheter system 100 herein can include from five light guides to fifteen light guides. In yet other embodiments, the catheter system 100 herein can include from ten light guides to thirty light guides. The catheter system 100 herein can include 1-30 light guides. It is appreciated that the catheter system 100 herein can include any number of light guides that can fall within a range, wherein any of the forgoing numbers can serve as the lower or upper bound of the range, provided that the lower bound of the range is a value less than the upper bound of the range. In some embodiments, the catheter system 100 herein can include greater than 30 light guides.

The manifold 136 can be positioned at or near the shaft proximal end 114. The manifold 136 can include one or more proximal end openings that can receive the one or more light guides, such as light guide 122, the guidewire 112, and/or an inflation conduit 140. The catheter system 100 can also include the fluid pump 138 that is configured to inflate the balloon 104 with the balloon fluid 132 and/or deflate the balloon 104 as needed.

As with all embodiments illustrated and described herein, various structures may be omitted from the figures for clarity and ease of understanding. Further, the figures may include certain structures that can be omitted without deviating from the intent and scope of the invention.

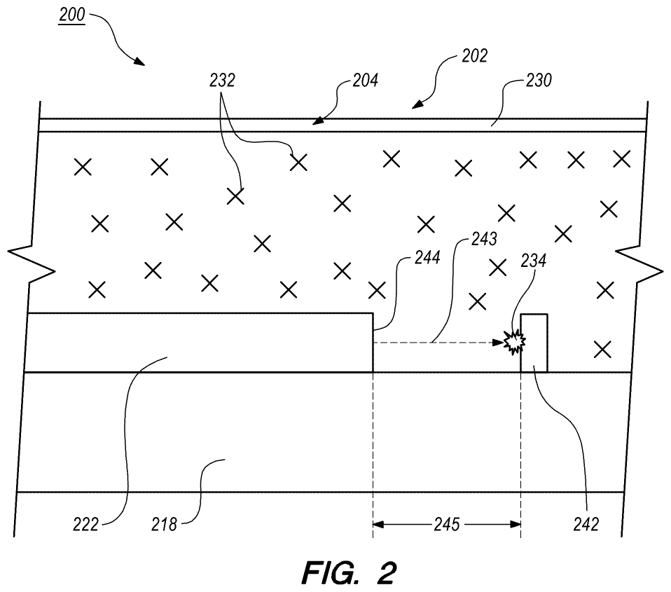

FIG. 2 is a simplified schematic side view of one embodiment of a portion of the catheter system 200, including one embodiment of a portion of a catheter 202. In the embodiment illustrated in FIG. 2, the catheter system can include one or more of an inflatable balloon 204, a guidewire lumen 218, and a light guide 222. Although the light guide 222 in FIG. 2 (as well as other embodiments shown and/or described herein) is illustrated as being positioned adjacent to the guidewire lumen 218, it is understood that in some embodiments, the light guide 222 can be positioned within the guidewire lumen 218 or the catheter shaft 110 (illustrated in FIG. 1), or the light guide 222 can be incorporated into a portion of the guidewire lumen 218 or the catheter shaft 110. In still other embodiments, the light guide 222 can be positioned away from the guidewire lumen 218 and/or the catheter shaft 110. In yet other embodiments, the guidewire lumen 218 can be omitted from the catheter system 200. It is further recognized that the structures with reference characters included in FIG. 2 (as well as other figures shown and described herein) are not necessarily drawn to scale for ease of viewing and/or understanding.

In the embodiment illustrated in FIG. 2, the catheter system 200 also includes a plasma target 242 that is spaced apart from the distal tip 244 of the light guide 222. The plasma target 242 can be formed from various materials. In some embodiments, the plasma target 242 can be formed from metallics and/or metal alloys having relatively high melting temperatures, such as tungsten, tantalum, molybdenum, niobium, platinum and/or iridium. Alternatively, the plasma target 242 can be formed from at least one of magnesium oxide, beryllium oxide, tungsten carbide, titanium nitride, titanium carbonitride, and titanium carbide. Still alternatively, the plasma target 242 can be formed from at least one of diamond CVD and diamond. In other embodiments, the plasma target 242 can be formed from a transition metal, an alloy metal, or a ceramic material. Yet alternatively, in some embodiments, plasma target 242 can be formed at least partially from a polymer, a polymeric material, and/or a plastic such as polyimide and nylon. Still alternatively, the plasma target 242 can be formed from any other suitable material(s). As provided in greater detail herein, the geometry, configuration, size, and/or shape of the plasma target 242 can also be varied to suit the design requirements of the catheter system 200.

In the embodiment illustrated in FIG. 2, the light guide 222 emits light energy 243 (illustrated in dashed lines in FIG. 2) from a distal tip 244 of the light guide 222 toward the plasma target 242. The plasma target 242 is spaced apart from the distal tip 244 of the light guide by a target gap distance 245. The target gap distance 245 can vary. For example, in various embodiments, the target gap distance 245 can be at least 1 μm, at least 10 μm, at least 100 μm, at least 1 mm, at least 2 mm, at least 3 mm, at least 5 mm or at least 1 cm. The target gap distance 245 can vary depending upon the size, shape, and/or angle of the plasma target 242 relative to the light energy emitted by the light guide 222, the type of material used to form the plasma target 242, the quantity and/or duration of the light energy being emitted from the light guide 222, the type of balloon fluid 232 used in the balloon 204, etc.

In certain embodiments, the plasma target 242 can be secured to another structure of the catheter system 200. For example, the plasma target 242 can be fixedly or movably secured or coupled to the guidewire lumen 218, as illustrated in FIG. 2. Alternatively, the plasma target 242 can be fixedly or movably secured or coupled to the light guide 222 or another suitable structure. Still, alternatively, the plasma target 242 can be suspended (unsecured) within the balloon fluid 232.

With this design, the light energy 243 generates a plasma bubble 234, which creates an outwardly emanating pressure wave (not shown) throughout the balloon fluid 232 that impacts the balloon 204. The impact to the balloon 204 causes the balloon to forcefully disrupt and/or fracture the vascular lesion, e.g., a calcified vascular lesion, at the treatment site 106 (illustrated in FIG. 1). In other words, the associated rapid formation of the plasma bubble 234 and resulting localized balloon fluid 232 velocity within the balloon 204 transfers mechanical energy though the incompressible balloon fluid 232 to impart a fracture force on the treatment site 106. The rapid change in momentum of the balloon fluid 232 upon hitting the balloon wall 230 is known as hydraulic shock, or water hammer. The change in momentum of the balloon fluid 232 is transferred as a fracture force to the vascular lesion, which is opposed to the balloon wall 230.

By positioning the plasma target 242 away from the distal tip 244 of the light guide 222, damage to the light guide 222 from the plasma bubble 234 is less likely to occur than if the plasma bubble 234 was generated at or more proximate to the distal tip 244 of the light guide. Stated another way, the presence of the plasma target 242, and positioning the plasma target 242 away from the distal tip 244 of the light guide 222, causes the plasma bubble 234 to, in turn, be generated away from the distal tip 244 of the light guide 222, reducing the likelihood of damage to the light guide 222. Further, in this embodiment, the positioning of the plasma target 242 can also be different from those previously described.

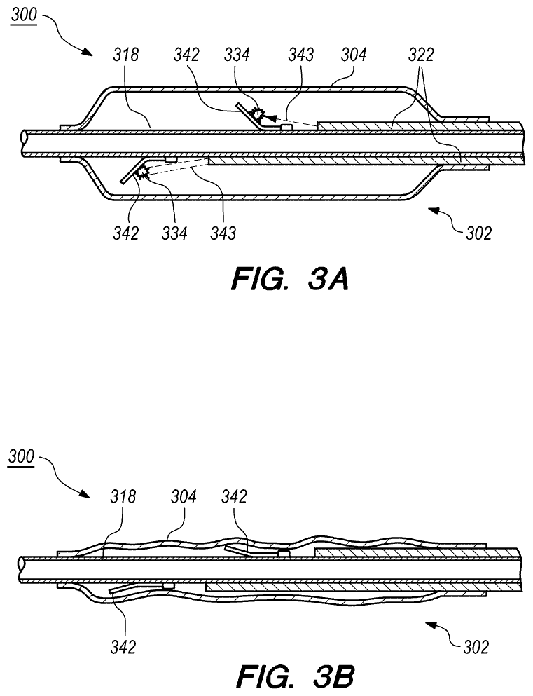

FIG. 3A is a simplified schematic side view of another embodiment of a portion of the catheter system 300, including another embodiment of a portion of the catheter 302, shown in an inflated state. In this embodiment, the catheter 302 includes a balloon 304, a guidewire lumen 318, one or more light guides 322 (two light guides 322 are illustrated in FIG. 3A), and one or more plasma targets 342 (two plasma targets 342 are illustrated in FIG. 3A). In the embodiment illustrated in FIG. 3A, the light guides 322 can be substantially similar to the light guides previously shown and described herein and/or shown in greater detail below.

However, in this embodiment, the plasma targets 342 can be movable depending upon the inflation status of the balloon 304. For example, the plasma targets 342 can include springs, e.g., can be spring-loaded, that extend outwardly toward the balloon 304 when the balloon 304 is in the inflated state. Stated another way, the plasma targets 342 can move and/or extend toward the balloon 304 (or in another suitable direction) so that the light energy 343 from the light guide(s) 322 is better directed toward the plasma target(s) 342. As previously described herein, because the plasma target 342 is positioned away from the distal tip 344 of the light guide 322, the plasma bubble 334 that is generated is less likely to cause damage to the light guide than if the plasma bubble 334 were generated at or more near to the light guide 322.

Further, in this embodiment, the positioning of the plasma targets 342 can be staggered (with two or more plasma targets 342) so that a greater area of the balloon 304 can be impacted by the resultant pressure wave(s) from the plasma bubbles 334.

FIG. 3B is a simplified schematic side view of the portion of the catheter illustrated in FIG. 3A, shown in a deflated state. In this embodiment, upon deflation of the balloon 304, the plasma targets 342 can retract or otherwise move back toward the guidewire lumen 318, or in another suitable direction, so that the catheter 302 can have a somewhat smaller diameter during insertion and/or removal of the catheter 302 from the blood vessel 108 (illustrated in FIG. 1), thereby increasing the ease of insertion and/or removal by the operator of the catheter system 300.

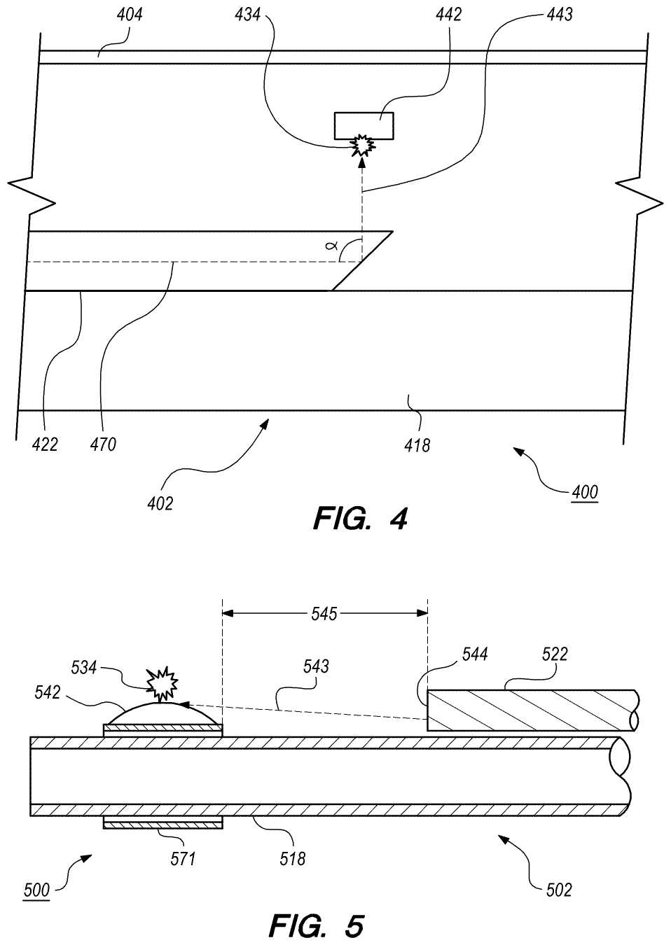

FIG. 4 is a simplified schematic side view of another embodiment of a portion of the catheter system 400, including another embodiment of a portion of the catheter 402. In the embodiment illustrated in FIG. 4, the catheter 402 includes a balloon 404, a guidewire lumen 418, one or more light guides 422, and one or more plasma targets 442.

The operation and function of the light guide 422 and the plasma target 442 can be substantially similar to those previously described. However, in this embodiment, the light guide 422 can be configured to redirect the light energy 443 in a different direction, i.e., non-parallel with a longitudinal axis 470 of the light guide 422. For example, the light energy can be redirected at an angle a relative to the longitudinal axis 470 of the light guide 422. In the embodiment illustrated in FIG. 4, the light energy 443 is redirected in a direction that is somewhat perpendicular to the longitudinal axis 470 of the light guide 422. However, it is understood that this type of angle is provided for ease of understanding only, and that any angle a between 0 and 180 degrees relative to the longitudinal axis 470 of the light guide 422 can be used. The structures and methods for redirecting the light energy 443 in this manner are provided in greater detail herein.

Further, in this embodiment, the positioning of the plasma target 442 can also be different from those previously described. For example, in one embodiment, the plasma target 442 is positioned between the light guide 422 and the balloon 404. In various embodiments, the plasma target 442 can be secured or coupled to another structure within the catheter 402, such as the guidewire lumen 418, the light guide 422, the balloon 404, or any other suitable structure. With this design, the plasma bubble 434 can be generated more proximate to the balloon 404, which can be beneficial for exerting a greater force to disrupt and/or fracture the calcified lesion and/or to maintain a spacing between the formation of the plasma bubble 434 and the light guide 422 for reasons provided herein.

FIG. 5 is a simplified cross-sectional view of another embodiment of a portion of the catheter system 500, including another embodiment of a portion of the catheter 502. In the embodiment illustrated in FIG. 5, the balloon has been omitted for clarity. In this embodiment, the catheter 502 includes a guidewire lumen 518, one or more light guides 522, one or more plasma targets 542, and a target coupler 571.

The operation and function of the light guide 522 and the plasma target 542 can be substantially similar to those previously described. However, in this embodiment, the plasma target 542 is coupled to the guidewire lumen 518 (or another suitable structure) with the target coupler 571. In one embodiment, the target coupler 571 can be a ring-like structure that secures the plasma target 542 to the guidewire lumen 518 (or another structure). The light energy 543 is emitted from the light guide 522, and results in a plasma bubble 534 being generated at the plasma target 542. Alternatively, the plasma target 542 can be adhered directly to the guidewire lumen 518 (or another structure) with adhesive or any other means for securing the plasma target 542. Still alternatively, the target coupler 571 can be movable so that the plasma target 542 can be moved either manually or automatically along the guidewire lumen 518 to change the target gap distance 545 between the plasma target 542 and the distal tip 544 of the light guide 522.

Additionally, or in the alternative, the shape of the plasma target 542 can vary. For example, in the embodiment illustrated in FIG. 5, the plasma target 542 can have a somewhat dome-shape or convex configuration. Still, alternatively, the plasma target 542 can have another suitable configuration. With these designs, the plasma bubble 534 can be generated at the plasma target 542, and the plasma target 542 can redirect the resultant pressure wave in any desired direction to achieve the desired results.

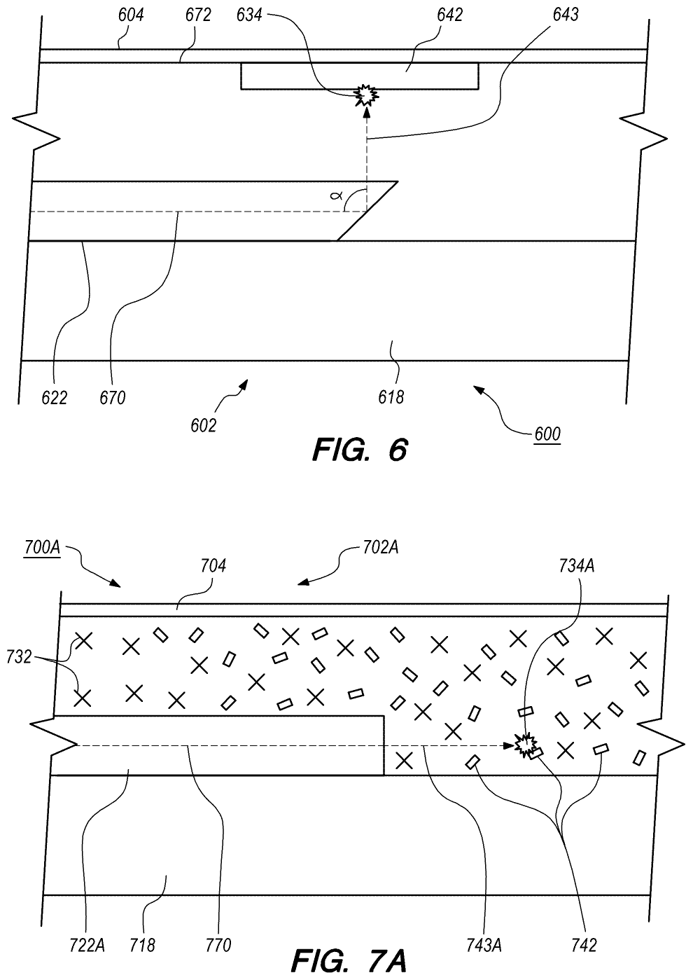

FIG. 6 is a simplified schematic side view of another embodiment of a portion of the catheter system 600, including another embodiment of a portion of the catheter 602. In the embodiment illustrated in FIG. 6, the catheter 602 includes a balloon 604, a guidewire lumen 618, one or more light guides 622, and one or more plasma targets 642.

The operation and function of the light guide 622 and the plasma target 642 can be substantially similar to those previously described. However, in this embodiment, the plasma target 642 is secured to a balloon inner surface 672 of the balloon 604. With this design, the plasma bubble is generated away from the light guide 622, thereby decreasing the likelihood of damage to the light guide 622. Moreover, because the plasma target 642 is positioned on the balloon inner surface 672, the balloon will receive a near-direct force from the plasma bubble 634 to increase the disruptive force upon the calcified lesion.

In this embodiment, the light guide 622 can be configured to redirect the light energy 643 in a different direction, i.e., non-parallel with a longitudinal axis 670 of the light guide 622. For example, the light energy can be redirected at an angle a relative to the longitudinal axis 670 of the light guide 622. In the embodiment illustrated in FIG. 6, the light energy 643 is redirected in a direction that is somewhat perpendicular to the longitudinal axis 670 of the light guide 622. However, it is understood that this type of angle is provided for ease of understanding only, and that any angle a between 0 and 180 degrees relative to the longitudinal axis 670 of the light guide 622 can be used. The structures and methods for redirecting the light energy 643 in this manner are provided in greater detail herein.

Further, in this embodiment, the positioning of the plasma target 642 can also be different from those previously described. For example, in one embodiment, the plasma target 642 is positioned between the light guide 622 and the balloon 604. In various embodiments, the plasma target 642 can be secured or coupled to another structure within the catheter 602, such as the guidewire lumen 618, the light guide 622, the balloon 604, or any other suitable structure. With this design, the plasma bubble 634 can be generated more proximate to the balloon 604, which can be beneficial for exerting a greater force to disrupt and/or fracture the calcified lesion and/or to maintain a spacing between the formation of the plasma bubble 634 and the light guide 622 for reasons provided herein.

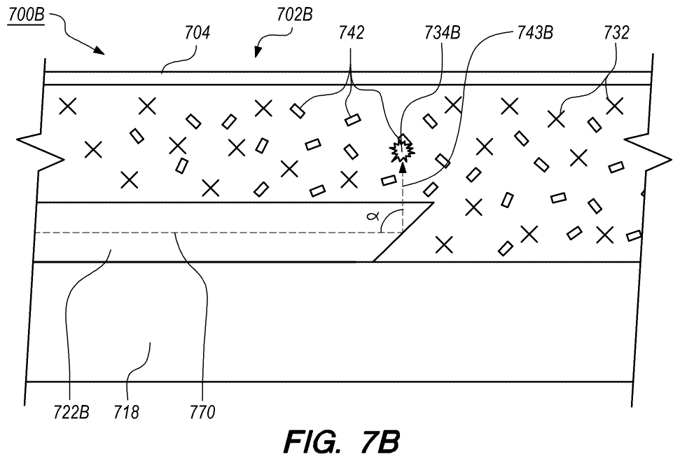

FIG. 7A is a simplified schematic side view of another embodiment of a portion of the catheter system 700A, including another embodiment of a portion of the catheter 702A. In the embodiment illustrated in FIG. 7A, the catheter 702A includes a balloon 704, a guidewire lumen 718, one or more light guides 722A, and a plurality of plasma targets 742.

The operation and function of the light guide 722A and the plasma targets 742 can be substantially similar to those previously described. However, in this embodiment, the light guide 722A can be configured to direct the light energy 743A parallel with a longitudinal axis 770 of the light guide 722B to generate plasma bubbles 734A at a plurality of plasma targets 742. In this embodiment, the plasma targets 742 are distributed throughout the balloon fluid 732 (illustrated as “X”'s in FIG. 7A). In this embodiment, the plasma targets 742 can be relatively small so that they can be suspended in the balloon fluid 732 more readily. In various embodiments, the plasma targets 742 can be free-floating within the balloon fluid 732, either as a homogeneous solution or a heterogeneous solution. With this design, one or more plasma bubbles 734A (only one plasma bubble 734A is illustrated in FIG. 7A) can be generated, which can be beneficial for exerting a greater force to disrupt and/or fracture the calcified lesion and/or to maintain a spacing between the formation of the plasma bubble 734A and the light guide 722A for reasons provided herein.

FIG. 7B is a simplified schematic side view of another embodiment of a portion of the catheter system 700B, including another embodiment of a portion of the catheter 702B. In the embodiment illustrated in FIG. 7B, the catheter 702B includes a balloon 704, a guidewire lumen 718, one or more light guides 722B, and a plurality of plasma targets 742.

The operation and function of the light guide 722B and the plasma targets 742 can be substantially similar to those previously described. However, in this embodiment, the light guide 722B can be configured to redirect the light energy 743B in a different direction, i.e., non-parallel with a longitudinal axis 770 of the light guide 722B. For example, the light energy 743B can be redirected at an angle a relative to the longitudinal axis 770 of the light guide 722B. In the embodiment illustrated in FIG. 7B, the light energy 743B is redirected in a direction that is somewhat perpendicular or orthogonal to the longitudinal axis 770 of the light guide 722B. However, it is understood that this type of angle is provided for ease of understanding only, and that any angle a between 0 and 180 degrees relative to the longitudinal axis 770 of the light guide 722B can be used. The structures and methods for redirecting the light energy 743B in this manner are provided in greater detail herein.

Further, in this embodiment, the positioning of the plasma targets 742 can also be different from those previously described. For example, in one embodiment, the plasma targets 742 are distributed throughout the balloon fluid 732 (illustrated as “X”'s in FIG. 7B). In this embodiment, the plasma targets 742 can be relatively small so that they can be suspended in the balloon fluid 732 more readily. In various embodiments, the plasma targets 742 can be free-floating within the balloon fluid 732, either as a homogeneous solution or a heterogeneous solution. With this design, one or more plasma bubbles 734B (only one plasma bubble 734B is illustrated in FIG. 7B) can be generated more proximate to the balloon 704, which can be beneficial for exerting a greater force to disrupt and/or fracture the calcified lesion and/or to maintain a spacing between the formation of the plasma bubble 734B and the light guide 722B for reasons provided herein.

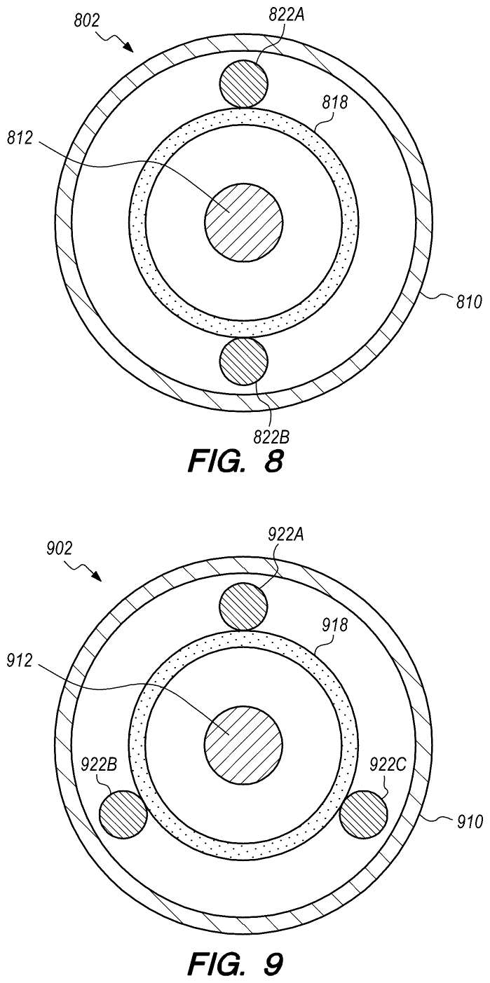

Examples of the catheters in accordance with the various embodiments herein include those having multiple light guides disposed about the catheter shaft at different positions around the circumference, as shown in FIGS. 8-11. It is understood that multiple light guides can be used with any of the embodiments shown and/or described herein without deviating from the intent and/or scope of the invention.

Referring now to FIG. 8, a schematic cross-sectional view of a catheter 102 in FIG. 1 along line 8-8 in FIG. 1 is shown in accordance with various embodiments herein. The catheter 802 illustrated in FIG. 8 can include one or more of a catheter shaft 810, a guidewire 812, a guidewire lumen 818, a first light guide 822A and a second light guide 822B separated by about 180 degrees around the circumference from the first light guide 822A. The first light guide 822A includes a side surface that can include any surface portion about a circumference of the first light guide 822A. The second light guide 822B includes a side surface that can include any surface portion about the circumference of the second light guide 822B. In some embodiments, the side surface spans a portion of the circumference of the light guides herein, such that it is less than cylindrical. In other embodiments, the side surface can span the entire circumference of the light guides herein such that it is cylindrical. It is recognized that any light guide described herein can include a side surface about the circumference of the light guide.

Referring now to FIGS. 9-11, schematic cross-sectional views of additional configurations for catheters having multiple light guides are shown in accordance with various embodiments herein. The embodiment of the catheter 902 illustrated in FIG. 3 can include one or more of a catheter shaft 910, a guidewire 912, a guidewire lumen 918, a first light guide 922A, a second light guide 922B, and a third light guide 922C separated by about 120 degrees around the circumference.

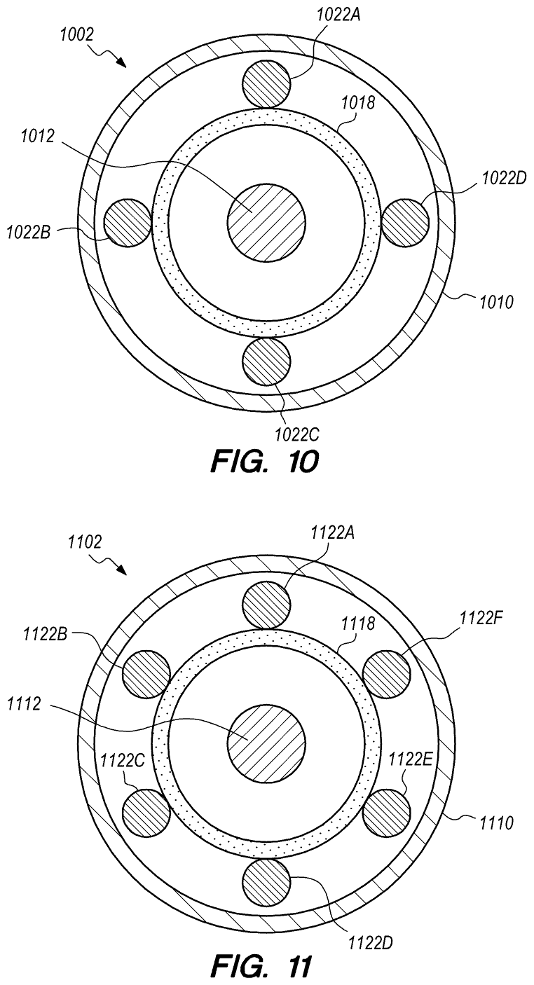

The embodiment of the catheter 1002 illustrated in FIG. 10 includes one or more of a catheter shaft 1010, a guidewire 1012, a guidewire lumen 1018, a first light guide 1022A, a second light guide 1022B, a third light guide 1022C, and a fourth light guide 1022D separated by about 90 degrees around the circumference.

The embodiment of the catheter 1102 illustrated in FIG. 11 includes one or more of a catheter shaft 1110, a guidewire 1112, a guidewire lumen 1118, a first light guide 1122A, a second light guide 1122B, a third light guide 1122C, a fourth light guide 1122D, a fifth light guide 1122E, and a sixth light guide 1122F separated by about 60 degrees around the circumference. It is understood that greater than six light guides can be used in the embodiments herein.

It is further appreciated that the light guides described herein can be disposed uniformly or nonuniformly about the catheter shaft to achieve the desired effect in the desired locations.

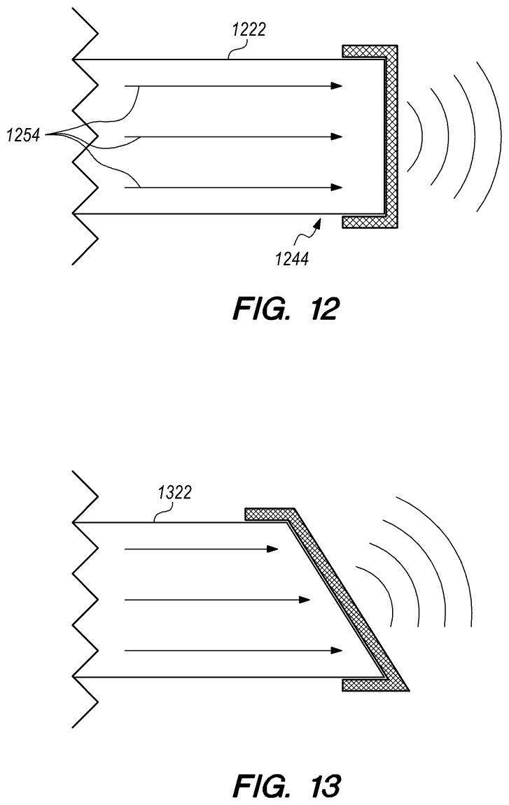

Diverting features and focusing features (also sometimes referred to herein simply as “diverting features”) will be discussed in more detail below and in reference to FIGS. 12-15. The light guides herein can include one or more diverting features, where each diverting feature can be in optical communication with the light guide within which it is disposed. In some embodiments, the diverting features can be in optical communication with a distal end of the light guide. Referring now to FIGS. 12-15, schematic cross-sectional views of the distal ends of various shaped light guides are shown in accordance with various embodiments herein.

In FIG. 12, a schematic cross-sectional view of a light guide 1222 is shown. Light guide 1222 is configured such that light 1254 travels from the power source 124 (illustrated in FIG. 1) in a direction from the shaft proximal end 114 (illustrated in FIG. 1) to the distal tip 1244, as indicated by arrows 1254.

In some embodiments, the end of the light guide can have an angled shape. By way of example, in FIG. 13 a schematic cross-sectional view of a light guide 1322 is shown.

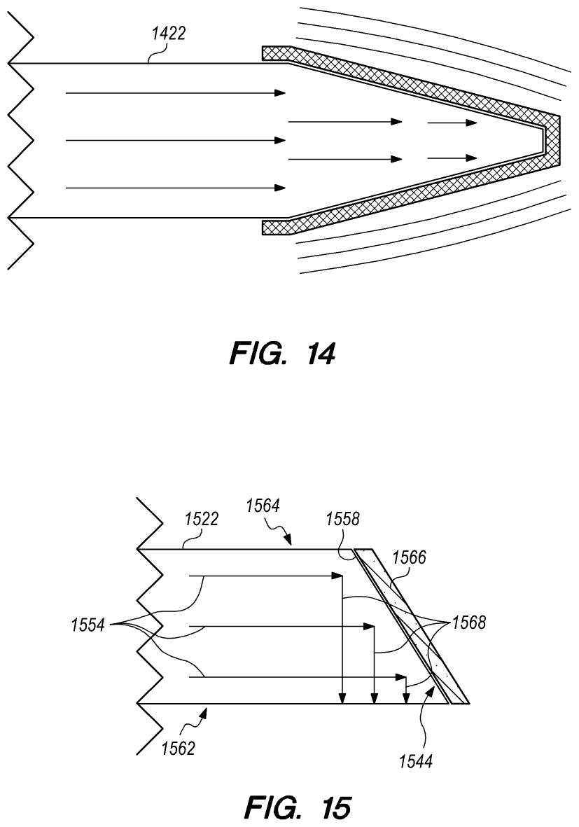

In some embodiments, the end of the light guide can have a tapered shape. By way of example, in FIG. 14 a schematic cross-sectional view of a light guide 1422 is shown.

Referring now to FIG. 15, a schematic cross-sectional view of a light guide 1522 is shown. Light guide 1522 includes an angled end 1558 disposed on a side surface 1562 of a distal end 1564 of the light guide 1522. The light guide 1522 includes a diverting feature 1566 at the distal tip 1544 to direct the light energy 1554 within the light guide 1522 toward the side surface 1562 of the light guide 1522. Light guide 1522 is configured such that light energy 1554 travels from the distal tip 1544 in a direction that is approximately 90 degrees (or another suitable angle) from the longitudinal axis 470 (illustrated in FIG. 4, for example) as indicated by arrows 1568. Upon contact with the diverting feature 1566, the light energy 1554 is diverted, or reflected, within the light guide 1522 to a side surface 1562 of the light guide 1522. The light energy 1554 extends away from the side surface 1562 of the light guide 1522.

The diverting feature 1566 of light guide 1522 can be made from a reflecting element or a refracting element. The diverting feature 1566 can be made from a glass, a polymer, a mirror, or a reflective metal coating. It is appreciated that the angle of internal reflection by the diverting feature 1566 can be adjusted by changing the angle of the distal tip 1544 of light guide 1522.

In some embodiments, a diverting feature can be included with the light guide to direct light toward a side surface of the distal end of the light guide. A diverting feature can include any feature of the system herein that diverts light from the light guide away from its axial path toward a side surface of the light guide. Examples include a reflector, a refracting structure, and a fiber diffuser.

In some embodiments herein, the light guides can include multiple diverting features. By way of example, each light guide herein can include a first diverting feature, a second diverting feature, a third diverting feature or a fourth diverting feature. In other embodiments, each light guide can include more than four diverting features. The diverting features can be configured to direct light to exit a light guide at a side surface thereof toward the balloon wall. In some examples, the diverting feature directs light toward the balloon surface closest to the diverting feature, so that the light does not cross the longitudinal axis of the catheter on its path to the balloon surface. It is appreciated that the diverting features can be in optical communication with corresponding light window.

The diverting features herein can be configured to direct light in the light guide toward a side surface of the distal portion, where the side surface is in optical communication with a light window. It is appreciated that the light guides herein can each include multiple diverting features and multiple light windows. Examples of the diverting features suitable for use herein include a reflecting element, a refracting element and/or a fiber diffuser.

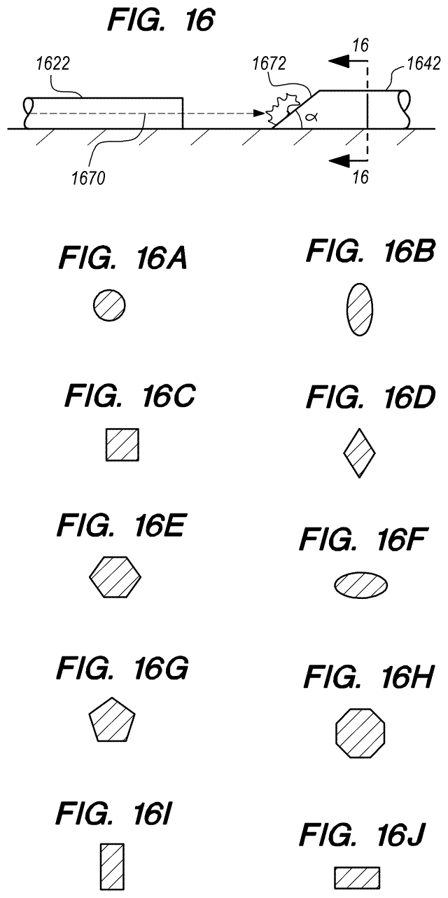

FIG. 16 is a simplified schematic side view of one embodiment of a portion of a catheter, including a light guide 1622 and an embodiment of a portion of a plasma target 1642. The plasma target 1642 includes a target face 1672. The target face 1672 can have any suitable geometry, shape or configuration. In this embodiment, the light guide 1622 and the plasma target 1642 operate substantially similar to those previously shown and/or described. However, in this embodiment, the target face 1672 is angled relative to a longitudinal axis 1670 of the light guide 1622. Stated another way, the target face 1672 has an angle a that can be any angle between 0 and 180 degrees relative to the longitudinal axis 1670 of the light guide 1622.

FIGS. 16A-16J are cross-sectional views showing representative, non-exclusive, non-limiting embodiments of the cross-sectional shape of the plasma target 1642. It is understood that there are literally an infinite number of possible cross-sectional configurations for the plasma target 1642 and that it would be an impossibility to show and describe all such configurations. However, it is the intent that the scope of this invention would encompass all such potential configurations, even those that are not shown and/or described herein.

FIG. 16A is a cross-sectional view of one embodiment of the plasma target 1642 taken on line 16-16 in FIG. 16. In this embodiment the plasma target 1642 has a substantially circular cross-sectional shape.

FIG. 16B is a cross-sectional view of another embodiment of the plasma target 1642 taken on line 16-16 in FIG. 16. In this embodiment the plasma target 1642 has a substantially vertical oval or elliptical cross-sectional shape.

FIG. 16C is a cross-sectional view of another embodiment of the plasma target 1642 taken on line 16-16 in FIG. 16. In this embodiment the plasma target 1642 has a substantially square cross-sectional shape.

FIG. 16D is a cross-sectional view of another embodiment of the plasma target 1642 taken on line 16-16 in FIG. 16. In this embodiment the plasma target 1642 has a substantially diamond, trapezoidal or parallelogram cross-sectional shape.

FIG. 16E is a cross-sectional view of another embodiment of the plasma target 1642 taken on line 16-16 in FIG. 16. In this embodiment the plasma target 1642 has a substantially hexagonal cross-sectional shape.

FIG. 16F is a cross-sectional view of another embodiment of the plasma target 1642 taken on line 16-16 in FIG. 16. In this embodiment the plasma target 1642 has a substantially horizonal oval or elliptical cross-sectional shape.

FIG. 16G is a cross-sectional view of another embodiment of the plasma target 1642 taken on line 16-16 in FIG. 16. In this embodiment the plasma target 1642 has a substantially pentagonal cross-sectional shape.

FIG. 16H is a cross-sectional view of another embodiment of the plasma target 1642 taken on line 16-16 in FIG. 16. In this embodiment the plasma target 1642 has a substantially octagonal cross-sectional shape.

FIG. 16I is a cross-sectional view of another embodiment of the plasma target 1642 taken on line 16-16 in FIG. 16. In this embodiment the plasma target 1642 has a substantially vertical rectangular cross-sectional shape.

FIG. 16J is a cross-sectional view of another embodiment of the plasma target 1642 taken on line 16-16 in FIG. 16. In this embodiment the plasma target 1642 has a substantially horizonal rectangular cross-sectional shape.

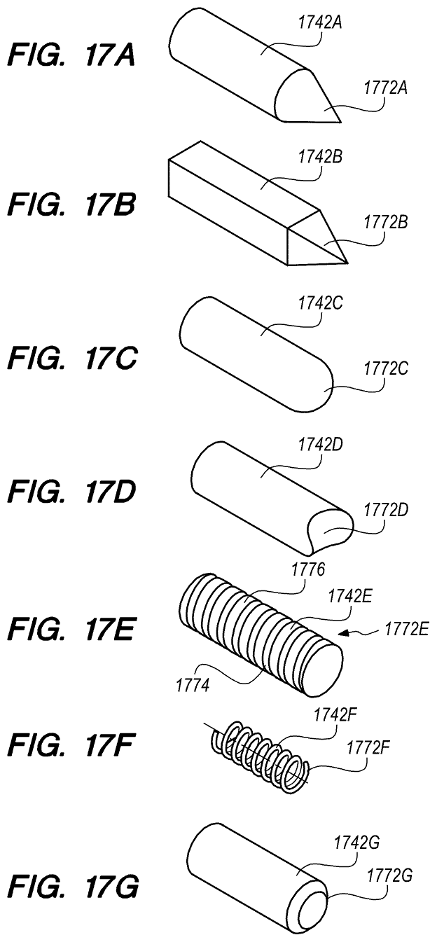

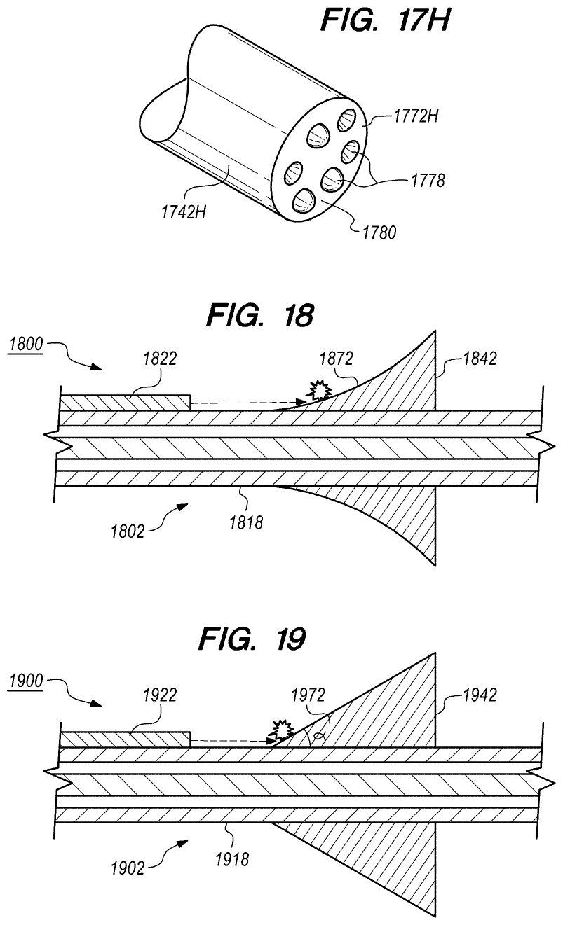

FIGS. 17A-17H are perspective views showing representative, non-exclusive, non-limiting embodiments of the geometry, shape and/or configuration of the target face 1772A-H of the plasma target 1742A-H. It is understood that there are literally an infinite number of possible cross-sectional configurations for the target face 1772A-H of the plasma target 1742A-H and that it would be an impossibility to show and describe all such configurations. However, it is the intent that the scope of this invention would encompass all such potential configurations, even those that are not shown and/or described herein.

FIG. 17A is a perspective view of a portion of an embodiment of the plasma target 1742A having one embodiment of a target face 1772A. In this embodiment, the target face 1772A has a somewhat conical configuration.

FIG. 17B is a perspective view of a portion of an embodiment of the plasma target 1742B having one embodiment of a target face 1772B. In this embodiment, the target face 1772B has a somewhat pyramidal configuration.

FIG. 17C is a perspective view of a portion of an embodiment of the plasma target 1742C having one embodiment of a target face 1772C. In this embodiment, the target face 1772C has a somewhat convex or dome-shaped configuration.

FIG. 17D is a perspective view of a portion of an embodiment of the plasma target 1742D having one embodiment of a target face 1772D. In this embodiment, the target face 1772D has a somewhat concave configuration.

FIG. 17E is a perspective view of a portion of an embodiment of the plasma target 1742E having one embodiment of a target face 1772E. In this embodiment, the target face 1772E includes a spiral projection 1774 that extends outwardly from a side portion 1776 of the plasma target 1742E.

FIG. 17F is a perspective view of a portion of an embodiment of the plasma target 1742F having one embodiment of a target face 1772F. In this embodiment, the target face 1772F has a somewhat spring-like or coiled configuration.

FIG. 17G is a perspective view of a portion of an embodiment of the plasma target 1742G having one embodiment of a target face 1772G. In this embodiment, the target face 1772G has a beveled configuration.

FIG. 17H is a perspective view of a portion of an embodiment of the plasma target 1742H having one embodiment of a target face 1772H. In this embodiment, the target face 1772H includes one or more surface features 1778. The surface features 1778 can include dimples, depressions or indentations that extend into a target surface 1780 of the target face 1772H. Additionally, or in the alternative, the surface features 1778 can include projections that extend outwardly from the target surface 1780 of the target face 1772H. In one embodiment, the surface features 1778 can include the same or other materials that are added to the target surface 1780. The specific sizes and/or shape(s) of the surface features 1778 can be varied.

FIG. 18 is a cross-sectional view of a portion of the catheter system 1800 including one embodiment of a portion of the catheter 1802. In this embodiment, the catheter 1802 can include a guidewire lumen 1818, one or more light guides 1822 and an embodiment of a portion of a plasma target 1842. The plasma target 1842 is spaced apart from the light guide 1822, and includes a target face 1872. The target face 1872 can have any suitable geometry, shape or configuration. In this embodiment, the light guide 1822 and the plasma target 1842 operate substantially similar to those previously shown and/or described. However, in this embodiment, the plasma target 1842 can be annular and can encircle the circumference of the guidewire lumen 1818. Further, the target face 1872 of the plasma target 1842 can have a somewhat concave, conical configuration. In alternative, non-exclusive embodiments, the plasma target 1842 can have a beveled, toroidal or frustoconical configuration, or any other suitable configuration. In an alternative embodiment, the plasma target 1842 only partially encircles the guidewire lumen 1818. Still alternatively, the plasma target 1842 can encircle or partially encircle another structure of the catheter 1802.

FIG. 19 is a cross-sectional view of a portion of the catheter system 1900 including one embodiment of a portion of the catheter 1902. In this embodiment, the catheter 1902 can include a guidewire lumen 1918, one or more light guides 1922 and an embodiment of a portion of a plasma target 1942. The plasma target 1942 is spaced apart from the light guide 1922, and includes a target face 1972. The target face 1972 can have any suitable geometry, shape or configuration. In this embodiment, the light guide 1922 and the plasma target 1942 operate substantially similar to those previously shown and/or described. However, in this embodiment, the plasma target 1942 can be annular and can encircle the circumference of the guidewire lumen 1918. Further, the target face 1972 of the plasma target 1942 can have a somewhat conical or pyramidal configuration. In an alternative embodiment, the plasma target 1942 only partially encircles the guidewire lumen 1918. Still alternatively, the plasma target 1942 can encircle or partially encircle another structure of the catheter 1902.

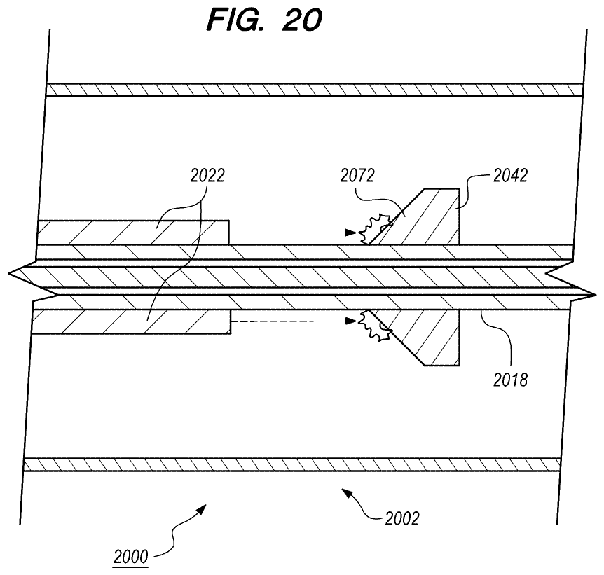

FIG. 20 is a cross-sectional view of a portion of the catheter system 2000 including one embodiment of a portion of the catheter 2002. In this embodiment, the catheter 2002 can include a guidewire lumen 2018, two or more light guides 2022 (only two light guides 2022 are illustrated in FIG. 20) and an embodiment of a portion of a plasma target 2042. The plasma target 2042 is spaced apart from the light guides 2022, and includes a target face 2072. The target face 2072 can have any suitable geometry, shape or configuration. In this embodiment, the light guides 2022 and the plasma target 2042 operate substantially similar to those previously shown and/or described. In an alternative embodiment, two or more plasma targets 2042 can be secured to the guidewire lumen 2018, wherein each only partially encircles the guidewire lumen 2018. Still alternatively, the plasma target 2042 can encircle or partially encircle another structure of the catheter 2002. It is appreciated that greater than two light guides 2022 can be used with the catheter system 2000 herein. For example, three light guides 2022 can be evenly spaced apart by 120 degrees from one another; four light guides 2022 can be evenly spaced apart by 90 degrees from one another, etc. Still alternatively, any number of light guides 2022 may be positioned so they are not evenly spaced circumferentially around the guidewire lumen 2018.

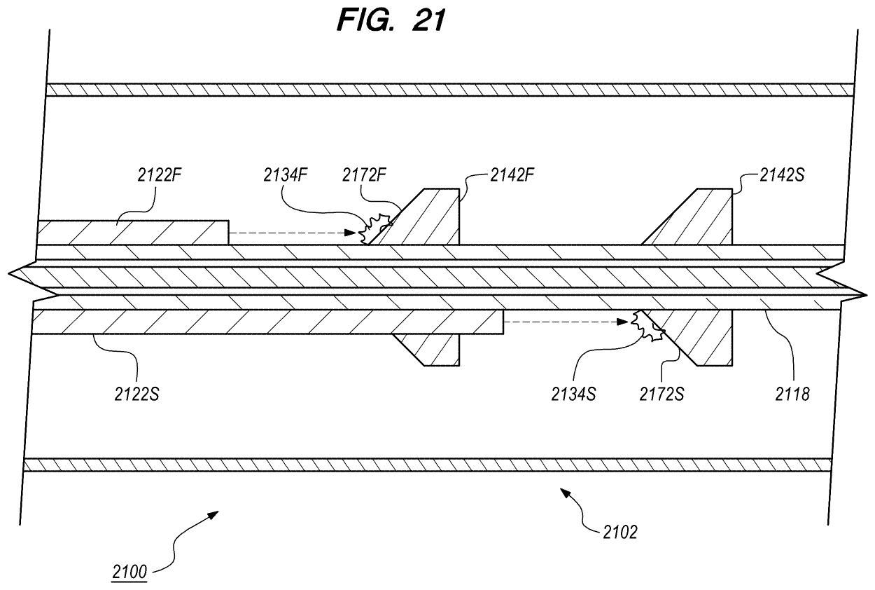

FIG. 21 is a cross-sectional view of a portion of the catheter system 2100 including one embodiment of a portion of the catheter 2102. In this embodiment, the catheter 2102 can include a guidewire lumen 2118, two or more light guides including a first light guide 2122F, and a second light guide 2122S (only two light guides are illustrated in FIG. 21) and two or more plasma targets including a first plasma target 2142F having a first target face 2172F, and a second plasma target 2142S (only two plasma targets are illustrated in FIG. 21) having a second target face 2172S. In the embodiment illustrated in FIG. 21, the first light guide 2122F emits light energy to generate a first plasma bubble 2134F at or near the first target face 2172F of the first plasma target 2142F. The second light guide 2122S emits light energy to generate a second plasma bubble 2134S at or near the second target face 2172S of the second plasma target 2142S, which is spaced apart from the first plasma target 2142F. In the embodiment illustrated in FIG. 21, the second light guide 2122S extends through the first plasma target 2142F. Alternatively, the second light guide 2122S can traverse around the first plasma target 2142F. Still alternatively, any or all of the plasma targets 2142F, 2142S can either encircle the guidewire lumen 2118, or partially encircle the guidewire lumen 2118.

### Balloons

The balloons suitable for use in the catheter systems illustrated and/or described herein include those that can be passed through the vasculature of a patient when in a collapsed configuration. In some embodiments, the balloons illustrated and/or described herein are made from silicone. In other embodiments, the balloons herein are made from polydimethylsiloxane (PDMS), polyurethane, polymers such as PEBAX™ material available from Arkema, which has a location at King of Prussia, Pennsylvania, USA, nylon, and the like. In some embodiments, the balloons can include those having diameters ranging from 1 millimeter (mm) to 25 mm in diameter. In some embodiments, the balloons can include those having diameters ranging from at least 1.5 mm to 12 mm in diameter. In some embodiments, the balloons can include those having diameters ranging from at least 1 mm to 5 mm in diameter. In some embodiments, the diameter can be greater than or equal to 0.5 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, 10.5 mm, 11.0 mm, 11.5 mm, 12.0 mm, 12.5 mm, 13.0 mm, 13.5 mm, 14.0 mm, 14.5 mm, 15.0 mm, 15.5 mm, 16.0 mm, 16.5 mm, 17.0 mm, 17.5 mm, 18.0 mm, 18.5 mm, 19.0 mm, 19.5 mm, or 20.0 mm, or can be an amount falling within a range between any of the foregoing.

In some embodiments, the balloons illustrated and/or described herein can include those having a length ranging from at least 5 mm to 300 mm in length. In some embodiments, the balloons illustrated and/or described herein can include those having a length ranging from at least 8 mm to 200 mm in length. In some embodiments, the length of the balloon can be greater than or equal to 5 mm, 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, 290 mm, or 300 mm, or can be an amount falling within a range between any of the foregoing.

The balloons illustrated and/or described herein can be inflated to inflation pressures from 1 atmosphere (atm) to 70 atm. In some embodiments, the balloons illustrated and/or described herein can be inflated to inflation pressures of from at least 20 atm to 70 atm. In some embodiments, the balloons illustrated and/or described herein can be inflated to inflation pressures of from at least 6 atm to 20 atm. In some embodiments, the balloons illustrated and/or described herein can be inflated to inflation pressures of from at least 3 atm to 20 atm. In some embodiments, the balloons illustrated and/or described herein can be inflated to inflation pressures of from at least 2 atm to 10 atm. In some embodiments, the balloons illustrated and/or described herein can be inflated to inflation pressures that can be greater than or equal to 1 atm, 2 atm, 3 atm, 4 atm, 5 atm, 6 atm, 7 atm, 8 atm, 9 atm, 10 atm, 15 atm, 20 atm, 25 atm, 30 atm, 35 atm, 40 atm, 45 atm, 50 atm, 55 atm, 60 atm, 65 atm, or 70 atm, or can be an amount falling within a range between any of the foregoing.

The balloons illustrated and/or described herein can include those having various shapes, including, but not to be limited to, a conical shape, a square shape, a rectangular shape, a spherical shape, a conical/square shape, a conical/spherical shape, an extended spherical shape, an oval shape, a tapered, shape, a bone shape, a stepped diameter shape, an offset shape, or a conical offset shape. In some embodiments, the balloons illustrated and/or described herein can include a drug eluting coating or a drug eluting stent structure. The drug elution coating or drug eluting stent can include one or more therapeutic agents including anti-inflammatory agents, anti-neoplastic agents, anti-angiogenic agents, and the like.

### Balloon Fluids

Exemplary balloon fluids suitable for use herein can include, but are not to be limited to one or more of water, saline, contrast medium, fluorocarbons, perfluorocarbons, gases, such as carbon dioxide, and the like. In some embodiments, the balloon fluids illustrated and/or described herein can be used as base inflation fluids, discussed elsewhere herein. In some embodiments, the balloon inflation fluids include a mixture of saline to contrast medium in a volume ratio of 50:50. In some embodiments, the balloon fluids include a mixture of saline to contrast medium in a volume ratio of 25:75. In some embodiments, the balloon fluids include a mixture of saline to contrast medium in a volume ratio of 75:25. The balloon fluids suitable for use herein can be tailored on the basis of composition, viscosity, and the like in order to manipulate the rate of travel of the pressure waves therein. The balloon fluids suitable for use herein are biocompatible. A volume of balloon fluid can be tailored by the chosen power source and the type of balloon fluid used.

In some embodiments, the contrast agents used in the contrast media herein can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C5F12).

The balloon fluids illustrated and/or described herein can include those that include absorptive agents that can selectively absorb light in the ultraviolet (e.g., at least 10 nanometers (nm) to 400 nm), visible region (e.g., at least 400 nm to 780 nm), and near-infrared region of the electromagnetic spectrum (e.g., at least 780 nm to 2.5 μm), or in the far-infrared region of the electromagnetic spectrum of at least 10 nm to 2.5 micrometers (μm). Suitable absorptive agents can include those with absorption maxima along the spectrum from at least 10 nm to 2.5 μm. In various embodiments, the absorptive agent can be those that have an absorption maximum matched with the emission maximum of the laser used in the catheter system. By way of non-limiting examples, various lasers described herein can include neodymium: yttrium-aluminum-garnet (Nd:YAG−emission maximum=1064 nm) lasers. holmium:YAG (Ho:YAG−emission maximum=2.1 μm) lasers, or erbium:YAG (Er:YAG−emission maximum=2.94 μm). In some embodiments, the absorptive agents used herein can be water soluble. In other embodiments, the absorptive agents used herein are not water soluble. In some embodiments, the absorptive agents used in the balloon fluids herein can be tailored to match the peak emission of the power source. Various power sources having emission wavelengths of at least 10 nanometers to 1 millimeter are discussed elsewhere herein.

In some embodiments, introduction of the balloon fluid causes the expansion of the balloon from a collapsed configuration to a first expanded configuration and from a first expanded configuration to a second further expanded configuration. In addition, or alternatively, the expansion of the balloon can be accomplished using a shape-memory material or other means.

### Light Guides

The light guides illustrated and/or described herein can include an optical fiber or flexible light pipe. The light guides illustrated and/or described herein can be thin and flexible and can allow light signals to be sent with very little loss of strength. The light guides illustrated and/or described herein can include a core surrounded by a cladding about its circumference. In some embodiments, the core can be a cylindrical core or a partially cylindrical core. The core and cladding of the light guides can be formed from one or more materials, including but not limited to one or more types of glass, silica, or one or more polymers. The light guides may also include a protective coating, such as a polymer. It is appreciated that the index of refraction of the core will be greater than the index of refraction of the cladding.

Each light guide can guide light along its length to a distal portion having at least one optical window. The light guides can create a light path as portion of an optical network including a power source. The light path within the optical network allows light to travel from one part of the network to another. Both the optical fiber or the flexible light pipe can provide a light path within the optical networks herein.

The light guides illustrated and/or described herein can assume many configurations about the catheter shaft of the catheters illustrated and/or described herein. In some embodiments, the light guides can run parallel to the longitudinal axis of the catheter shaft of the catheter. In some embodiments, the light guides can be disposed spirally or helically about the longitudinal axis of the catheter shaft of the catheter. In some embodiments, the light guides can be physically coupled to the catheter shaft. In other embodiments, the light guides can be disposed along the length of the outer diameter of the catheter shaft. In yet other embodiments the light guides herein can be disposed within one or more light guide lumens within the catheter shaft. Various configurations for the catheter shafts and light guide lumens will be discussed below.

### Diverting Features and Focusing Features

The diverting features suitable for use herein include a reflecting element, a refracting element, and a fiber diffuser. In some embodiments, the diverting feature can be a reflecting element. In some embodiments, the diverting feature can be a refracting element. In some embodiments, the diverting feature can be a fiber diffuser.

A fiber diffuser can direct light from within a light guide to exit at a side surface of the light guide. The fiber diffusers described herein can be created several ways. In some embodiments, the fiber diffusers can be created by micro-machining the surface of the distal portion of a light guide with a CO₂ laser. In some embodiments, a fused silica coating can be applied to the distal portion of the light guide. In other embodiments, the fiber diffuser can be formed from a glass, a polymer, or a metal coating on the distal portion of the light guide. In other embodiments, the fiber diffuser can be formed by a fiber Bragg grating on the distal portion of the light guide. In some embodiments, the fiber diffuser can include a machined portion of the light guide, a laser-machined portion of the light guide, fiber Bragg gratings, a fused splicing, a fused splicing forming at least one internal mirror, and a splicing of two or more diffuse regions.

Suitable materials for a fiber diffuser can include, but are not be limited to, the materials of the light guide core or light guide cladding, ground glass, silver coated glass, gold coated glass, TiO2, and other materials that will scatter and not significantly absorbed the light wavelength of interest. One method that can be used to create a uniform diffuser in a light guide, optical component, or materials is to utilize scattering centers on the order of at least 50 nanometers to 5 micrometers in size. The scattering centers can have a distribution about 200 nanometers in size.

The diverting features and focusing features suitable for focusing light away from the tip of the light guides herein can include, but are not to be limited to, those having a convex surface, a gradient-index (GRIN) lens, and a mirror focus lens.

### Power Sources

The power sources suitable for use herein can include various types of power sources including lasers and lamps. Suitable lasers can include short pulse lasers on the sub-millisecond timescale. In some embodiments, the power source can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths and energy levels that can be employed to achieve plasma in the balloon fluid of the catheters illustrated and/or described herein. In various embodiments, the pulse widths can include those falling within a range including from at least 10 ns to 200 ns. In some embodiments, the pulse widths can include those falling within a range including from at least 20 ns to 100 ns. In other embodiments, the pulse widths can include those falling within a range including from at least 1 ns to 5000 ns.

Exemplary nanosecond lasers can include those within the UV to IR spectrum, spanning wavelengths of about 10 nanometers to 1 millimeter. In some embodiments, the power sources suitable for use in the catheter systems herein can include those capable of producing light at wavelengths of from at least 750 nm to 2000 nm. In some embodiments, the power sources can include those capable of producing light at wavelengths of from at least 700 nm to 3000 nm. In some embodiments, the power sources can include those capable of producing light at wavelengths of from at least 100 nm to 10 micrometers (μm). Nanosecond lasers can include those having repetition rates of up to 200 KHz. In some embodiments, the laser can include a Q-switched thulium: yttrium-aluminum-garnet (Tm:YAG) laser. In some embodiments, the laser can include a neodymium: yttrium-aluminum-garnet (Nd:YAG), holmium: yttrium-aluminum-garnet (Ho:YAG), erbium: yttrium-aluminum-garnet (Er:YAG), excimer laser, helium-neon laser, carbon dioxide laser, as well as doped, pulsed, fiber lasers.

### Pressure Waves

The catheters illustrated and/or described herein can generate pressure waves having maximum pressures in the range of at least 1 megapascal (MPa) to 100 MPa. The maximum pressure generated by a particular catheter will depend on the power source, the absorbing material, the bubble expansion, the propagation medium, the balloon material, distance of measurement from plasma epicenter, and other factors. In some embodiments, the catheters illustrated and/or described herein can generate pressure waves having maximum pressures in the range of at least 2 MPa to 50 MPa. In other embodiments, the catheters illustrated and/or described herein can generate pressure waves having maximum pressures in the range of at least 2 MPa to 30 MPa. In yet other embodiments, the catheters illustrated and/or described herein can generate pressure waves having maximum pressures in the range of at least 15 MPa to 25 MPa. In some embodiments, the catheters illustrated and/or described herein can generate pressure waves having peak pressures of greater than or equal to 1 MPa, 2 MPa, 3 MPa, 4 MPa, 5 MPa, 6 MPa, 7 MPa, 8 MPa, 9 MPa, 10 MPa, 11 MPa, 12 MPa, 13 MPa, 14 MPa, 15 MPa, 16 MPa, 17 MPa, 18 MPa, 19 MPa, 20 MPa, 21 MPa, 22 MPa, 23 MPa, 24 MPa, or 25 MPa, 26 MPa, 27 MPa, 28 MPa, 29 MPa, 30 MPa, 31 MPa, 32 MPa, 33 MPa, 34 MPa, 35 MPa, 36 MPa, 37 MPa, 38 MPa, 39 MPa, 40 MPa, 41 MPa, 42 MPa, 43 MPa, 44 MPa, 45 MPa, 46 MPa, 47 MPa, 48 MPa, 49 MPa, or 50 MPa. It is appreciated that the catheters illustrated and/or described herein can generate pressure waves having operating pressures or maximum pressures that can fall within a range, wherein any of the forgoing numbers can serve as the lower or upper bound of the range, provided that the lower bound of the range is a value less than the upper bound of the range.

Therapeutic treatment can act via a fatigue mechanism or a brute force mechanism. For a fatigue mechanism, operating pressures would be about at least 0.5 MPa to 2 MPa, or about 1 MPa. For a brute force mechanism, operating pressures would be about at least 20 MPa to 30 MPa, or about 25 MPa. Pressures between the extreme ends of these two ranges may act upon a treatment site using a combination of a fatigue mechanism and a brute force mechanism.

The pressure waves described herein can be imparted upon the treatment site from a distance within a range from at least 0.1 millimeters (mm) to 25 mm extending radially from a longitudinal axis of a catheter placed at a treatment site. In some embodiments, the pressure waves can be imparted upon the treatment site from a distance within a range from at least 10 mm to 20 mm extending radially from a longitudinal axis of a catheter placed at a treatment site. In other embodiments, the pressure waves can be imparted upon the treatment site from a distance within a range from at least 1 mm to 10 mm extending radially from a longitudinal axis of a catheter placed at a treatment site. In yet other embodiments, the pressure waves can be imparted upon the treatment site from a distance within a range from at least 1.5 mm to 4 mm extending radially from a longitudinal axis of a catheter placed at a treatment site. In some embodiments, the pressure waves can be imparted upon the treatment site from a range of at least 2 MPa to 30 MPa at a distance from 0.1 mm to 10 mm. In some embodiments, the pressure waves can be imparted upon the treatment site from a range of at least 2 MPa to 25 MPa at a distance from 0.1 mm to 10 mm. In some embodiments, the pressure waves can be imparted upon the treatment site from a distance that can be greater than or equal to 0.1 mm, 0.2 mm, 0.3 mm, 0.4 mm, 0.5 mm, 0.6 mm, 0.7 mm, 0.8 mm, or 0.9 mm, 1 mm, 2 mm, 3 mm, 4 mm, 5 mm, 6 mm, 7 mm, 8 mm, 9 mm, or 10 mm, or can be an amount falling within a range between any of the foregoing.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

As used herein, the recitation of numerical ranges by endpoints shall include all numbers subsumed within that range, inclusive (e.g., 2 to 8 includes 2, 2.1, 2.8, 5.3, 7, 8, etc.).

It is recognized that the figures shown and described are not necessarily drawn to scale, and that they are provided for ease of reference and understanding, and for relative positioning of the structures.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

1. A catheter system for treating a treatment site within or adjacent to a blood vessel, the catheter system comprising:
a power source;
a light guide that receives power from the power source, the light guide having a distal tip, the light guide emitting light energy in a direction away from the distal tip;
an inflatable balloon that encircles the distal tip of the light guide;
a guidewire lumen that is positioned at least partially within the inflatable balloon; and
a plasma target that is secured to one of the guidewire lumen and the inflatable balloon, the plasma target being spaced apart from the distal tip of the light guide by a target gap distance, the plasma target being positioned within the inflatable balloon, the plasma target being configured to receive light energy from the light guide so that a plasma bubble is generated at the plasma target upon receiving the light energy from the light guide.

2. The catheter system of claim 1 wherein the plasma target is at least partially formed from one of tungsten, tantalum, platinum, molybdenum, niobium, and iridium.

3. The catheter system of claim 2 wherein the plasma target is further partially formed from one of a polymer and a polymeric material.

4. The catheter system of claim 2 wherein the plasma target is further partially formed from a ceramic material.

5. The catheter system of claim 1 wherein the plasma target is at least partially formed from one of magnesium oxide, beryllium oxide, tungsten carbide, titanium nitride, titanium carbonitride and titanium carbide.

6. The catheter system of claim 1 wherein the target gap distance is greater than 1 μm.

7. The catheter system of claim 1 wherein the target gap distance is greater than 100 μm.

8. The catheter system of claim 1 wherein the power source is a laser, and the light guide is an optical fiber.

9. The catheter system of claim 1 wherein the light guide is coupled to the guidewire lumen.

10. The catheter system of claim 1 wherein the plasma target is secured to the guidewire lumen.

11. The catheter system of claim 10 wherein the plasma target at least partially encircles the guidewire lumen.

12. The catheter system of claim 10 wherein the plasma target is mechanically coupled to the light guide.

13. The catheter system of claim 10 wherein the light guide includes a distal region having a longitudinal axis, and wherein the direction the light energy is emitted is substantially along the longitudinal axis of the distal region.

14. The catheter system of claim 10 wherein the plasma target has a target face that receives the light energy from the light guide, the target face being angled relative to a direction the light energy is emitted to the plasma target.

15. The catheter system of claim 14 wherein the target face includes one or more surface features.

16. The catheter system of claim 15 wherein the one or more surface features includes at least one of an indentation and a projection.

17. The catheter system of claim 14 wherein the target face includes one of a beveled edge, a conical configuration, a pyramidal configuration, a dome-shaped configuration, a concave configuration, a convex configuration, a multi-faceted configuration, a coiled configuration, a spring-like configuration, and a somewhat spiral configuration.

18. The catheter system of claim 10 wherein the plasma target is spring-loaded.

19. The catheter system of claim 1 wherein the plasma target is secured to the balloon.

20. The catheter system of claim 19 wherein the light guide includes a distal region having a longitudinal axis, and wherein the direction the light energy is emitted is angled relative to the longitudinal axis of the distal region.
